(19) United States
(12) Patent Application Publication
(10) Pub. No.: US 2009/0169623 A1
(43) Pub. Date: Jul. 2, 2009

(54) Use of ginsenosides and extracts containing them

(76) Inventors: Gerard Sene, Paris (FR); Alain Loiseau, Bouillon (FR); Alain Meybeck, Courbevoie (FR); Chong Ren Yang, Yunnan (CN)

Correspondence Address: Barbara A. Shimei;Director, Patents & Licensing, Bayer HealthCare LLC - Pharmaceuticals, 555 White Plains Road, Third Floor, Tarrytown, NY, 10591, US

(21) Appl. No.: 12/291,782

(22) Filed: Nov. 13, 2008

(30) Foreign Application Priority Data
May 17, 2006 (EP) EP 06290814.0

(51) Int. Cl. A61K 9/28; A61K 8/60; A61K 31/70; A61K 36/00

(52) U.S. Cl. 424/474; 424/59; 514/23; 424/725

(57) ABSTRACT

The present invention relates to the use of ginsenosides and a plant extract containing ginsenosides in a cosmetic or pharmaceutical composition or in a food supplement for the protection of the skin against deleterious effects of stress or irradiation e.g. sunlight or UV irradiation.

The present invention relates to the use of ginsenosides and a plant extract containing ginsenosides in a cosmetically or pharmaceutical composition or in a food supplement for the protection of the skin against deleterious effects of stress or irradiation such as sunlight or UV irradiation.

The Panax plant family comprises numerous species such as Panax ginseng (C. A. Meyer), Panax quinquefolium, and Panax notoginseng which are cultivated and used industrially in food supplements, pharmaceuticals and cosmetics. They contain saponins as active substances which are called ginsenosides. Ginsenosides of each species are basically the same, but are contained in different proportions in each species.

Panax notoginseng (also called San Qi) is a straight herbaceous perennial plant which grows e.g. in the southwest of China. Traditionally people use the roots of Panax notoginseng not only as a tonic but also for the treatment of many symptoms and diseases such as trauma, inflammation, hepatitis, heart and vascular diseases as well as aging. Up to now more than 30 ginsenosides could be isolated from the roots of Panax notogingseng. The major ginsenosides are ginsenoside Rg1 and ginsenoside Rb1 followed by ginsenosides Rd, Re, Rg2 and notoginsenoside R1. Many scientific works have confirmed the pharmacological properties and biological activities of Panax notoginseng such as ditropism regulating effects to organic function (Y. M. Luo et al., Acta Pharmacologica Sinica, 1993, 14(5), 401-404), effects on the central nervous system (Y. Ying et al., Acta Pharmaceutica Sinica, 1994, 29(4), 241-245), cancer prevention (T. Konoshima et al., Chemical and Pharmaceutical Bulletin, 1992, 40, 531-533; L. Xu et al., Journal of WCUMS, 1991, 22(2), 124-127) and antiviral activity (J. Li et al., Journal of Norman Bethune University of Medical Sciences, 1992, 18(1), 24-26).

In the cosmetic industry, Panax notoginseng and/or ginsenosides are used for various applications. Panax notoginseng extract is claimed for skin elasticity activation to improve wrinkles and prevent chronological and UV-induced aging (JP 2006-028150).

Ginsenoside Rb1 or Rb-1 like substances are described for stimulation of elastin synthesis (WO 99/07338), for treating hair (FR 9300899, U.S. Pat. No. 5,663,160), for stimulating the regeneration of tissues after the wound (JP 2002-255826), for the reconstruction of tissues suffering from skin aging (WO 2002/072599), for treating wounds in the case of burns (JP 2004-077456). Ginsenoside Rb1 is also used in combination with Ginsenosides Rc and Rd (JP 2003-070496) to activate endonuclease for the repair of UV damaged DNA. Ginsensosides Rh2 and Rg3 are blended in a UV-blocking cosmetic composition due to their UV absorbing properties (KR 2004-0098177).

Langerhans cells are responsible for the immune protection of skin. When skin is exposed to UV light, Langerhans cells disappear from the epidermis and as a result it becomes less protected against infectious diseases and cancer (T. Schwarz, Photodermatol Photoimmunol Photomed 2002, 18, 141-145).

Heme oxygenase HO is an enzyme which catalyzes the ring opening of heme (a molecule found in cells) with the formation of carbon monoxide CO, biliverdin (which is rapidly transformed into the antioxidant bilirubin) and free iron (which leads to the induction of the iron-binding protein ferritin). Heme oxygenase has two forms: HO-2 which is the constitutive form mainly in neural tissues and HO-1 which is the inducible form. They are considered to be cytoprotective enzymes due to the antioxidant, anti-inflammatory, anti-apoptotic and anti-proliferative effects (L. E. Otterbein et al., Trends Immunol. 2003, 24(8), 449-55). HO-1 induction is considered to improve burn injury healing (Gan HT et al., Surgery., 2006, 141(3):385-93) and its induction by 20(S)-Protopanaxadiol is proved to decrease NO production for anti-inflammatory activity (Lee SH et al., Planta Med., 2005, 71(12), 1167-70). It has also been found that carbon monoxide generated by HO-1, which itself is generated by UV-A exposure, can protect Langerhans cells from photoimmunosuppression caused by UV-B irradiation (M. Allanson et al., J. Invest. Dermatol., 2005, 124(3), 644-650).

The present invention relates to ginsenosides and/or a plant extracts containing them for the protection of the skin against deleterious effects of stress or irradiation e.g. sunlight or UV irradiation.

The use of ginsenosides and the extracts containing them according to the invention are an appropriate and safe method for the protection of the skin.

According to the invention ginsenosides include but are mot limited to the ginsenosides G-Ra1, G-Ra2, G-Ra3, G-Rb1, G-Rb2, G-Rb3, G-Rc, G-Rd, G-Re, G-Rf, G-Rg1, G-Rg2, G-Rg3, G-Rh1, G-Rh2, G-Rs1, G-Rs2, G-Ro, MG-Rb1, MG-Rb2, MG-Rc, MG-Rd, Q-R1, N-R1, N-R4 and 20 Glc-Rf. Preference is given to ginsenoside G-Rg1, G-Rb1, G-Rd, G-Re, G-Rg2 and N-R1. Most preferably the ginsenoside is selected from the group consisting of ginsenoside G-Rg1 and G-Rb1.

All mentioned ginsenosides are known and can be isolated from the Panax plants by standard methods e.g. by extraction and chromatography.

According to the invention ginsenosides refers to a single ginsenoside as well as to a mixture of different ginsenosides. Preference is given to a mixture of ginsenosides comprising ginsenoside G-Rg1 and G-Rb 1, more preferably a mixture of ginsenoside G-Rg1, G-Rb 1, G-Rd, G-Re, G-Rg2 and N-R1.

Plant extracts containing ginsenosides according to the invention are extracts of plants of the Panax family which include but are not limited to Panax ginseng (C. A. Meyer), Panax quinquefolium, and Panax notoginseng (San Qi). Preference is given to Panax notoginseng (San Qi).

The extraction can be performed on all parts of the plant. Preferably the roots or rhizomes are extracted.

The extraction can be done by standard extraction methods. Preferably roots or rhizomes are extracted with a polar solvent applicable for extraction optionally by several times. The crude extract can be purified by chromatography. Optionally the fractions can be dried by spray-drying.

An extract according to the invention is normally a dry extract. Nevertheless the extract can also be used as solution, i.e. that the final drying step of the described extraction process is omitted and the product can optionally be encapsulated, e.g. in a gelatine capsule

The polar solvent used for extraction is preferably alcohol or a mixture of water and alcohol wherein the alcohol is preferably ethanol.

Preference is given to a dry plant extract containing ginsenosides in an amount between 0.1 and 100%, preferably between 10 and 100%, preferably above 80%.

The extract according to the invention preferably contains G-Rb1 in an amount of from 10 to 60%, G-Rg1 in an amount of from 10 to 60%, G-Rd in an amount of from 0 to 15%, N-R1 in an amount of from 0 to 15% and/or G-Re in an amount of from 0 to 10% by weight of the total extract.

Ginsenosides can be isolated and/or purified from the extract containing it by standard isolation methods. Standard isolation methods include but are not limited to chromatographic methods.

Ginsenosides or the extract containing them according to the invention can be administered in any form by any effective route, including, e.g., oral, parenteral, enteral, intravenous, intraperitoneal, topical, transdermal (e.g., using any standard patch), ophthalmic, nasally, local, non-oral, such as aerosal, inhalation, subcutaneous, intramuscular, buccal, sublingual, rectal, vaginal, intra-arterial, and intrathecal, etc. They can be administered alone, or in combination with any ingredient(s), active or inactive. Preference is given to a topical or orally administration.

Ginsenosides or the extract containing them according to the invention can be converted in a known manner into the usual formulations such as cosmetically, dermatological, pharmaceutical or food supplement compositions. These may be liquid or solid formulations e.g. without limitation normal and enteric coated tablets, capsules, pills, powders, granules, elixirs, tinctures, solution, suspensions, suppositories, syrups, solid and liquid aerosols, emulsions, pastes, creams, ointments, milks, gels, salves, serums, foams, shampoos, sticks or lotions.

Preference is given to a dermatological or cosmetic composition in a form of an aqueous solution, a white or colored cream, ointment, milk, gel, salve, serum, foam, shampoo, stick, cream, paste, or lotion.

Also preference is given to an orally applicable food supplement composition comprising ginsenosides or the extract containing them according to the invention.

Ginsenosides or the extract containing them according to the invention can be further combined with any other suitable additive or pharmaceutically acceptable carrier, preferably dermatological and/or cosmetically acceptable carrier. Such additives include any of the substances already mentioned, as well as any of those used conventionally, such as those described in Remington: The Science and Practice of Pharmacy (Gennaro and Gennaro, eds, 20th edition, Lippincott Williams & Wilkins, 2000); Theory and Practice of Industrial Pharmacy (Lachman et al., eds., 3rd edition, Lippincott Williams & Wilkins, 1986); Encyclopedia of Pharmaceutical Technology (Swarbrick and Boylan, eds., 2nd edition, Marcel Dekker, 2002). These can be referred to herein as “pharmaceutically acceptable carriers” to indicate they are combined with the active drug and can be administered safely to a subject for therapeutic purposes.

The dosage of ginsenosides or the extract containing them of the present invention can be selected with reference to the effects to be treated and/or the type of disease and/or the disease status in order to provide the desired therapeutic activity. These amounts can be determined routinely for a particular patient, where various parameters are utilized to select the appropriate dosage (e.g., type of disease, age of patient, disease status, patient health, weight, etc.), or the amounts can be relatively standard.

The amount of the administered active ingredient can vary widely according to such considerations as the particular compound and dosage unit employed, the mode and time of administration, the period of treatment, the age, sex, and general condition of the patient treated, the nature and extent of the condition treated, the rate of drug metabolism and excretion, the potential drug combinations and drug-drug interactions, and the like.

Preference is given to a composition containing an extract according to the invention in an amount of more than 0.01% up to 10% by weight of the total composition.

Furthermore the composition according to the invention preferably contains G-Rb1 in an amount of from 0.001 to 6%, G-Rg1 in an amount of from 0.001 to 6%, G-Rd in an amount of from 0 to 1.5%, N-R1 in an amount of from 0 to 1.5% and/or G-Re in an amount of from 0 to 1% by weight of the total composition.

The composition according to the invention is administered one or more, preferably up to three, more preferably up to two times per day. Preference is given to a topical or orally administration.

Nevertheless, it may in some cases be advantageous to deviate from the amounts specified, depending on body weight, individual behaviour toward the active ingredient, type of preparation and time or interval over which the administration is effected. For instance, less than the aforementioned minimum amounts may be sufficient in some cases, while the upper limit specified has to be exceeded in other cases. In the case of administration of relatively large amounts, it may be advisable to divide these into several individual doses over the day.

Ginsenosides or the extract containing them according to the invention can also be combined with at least one further active substance or plant extract e.g. substances or plant extracts usually employed for dermatological use.

Further active substances include but are not limited to desquamating and/or moisturizing agents, UV filtering or blocking agents, depigmenting or propigmenting agents, antiglycation agents, anti-inflammatory agents, anti-microbial agents, agents stimulating the synthesis of dermal, epidermal, hair or nail macromolecules and/or preventing the degradation thereof, agents stimulating the differentiation of keratinocytes, muscle relaxants, antipollution and/or anti-free radical agents, slimming agents, agents acting on the microcirculation, agents acting on the energy metabolism of the cells, tightening agents, agents preventing the loss or stimulating the growth of hair, agents preventing grey or white hair, or a mixture thereof. Preferably that combination is contained in a topically dermatological or cosmetically composition.

Ginsenosides or the extract containing them according to the invention can also be combined with an alpha-hydroxy acid, a salicylic acid or derivatives thereof such as acetylsalicylic acid, a cystein derivative, a ceramide, a steroid, tocopherol, tocotrienol, arbutin or derivatives thereof, ascorbic acid or a derivative thereof, retinol or derivatives thereof, retinoid or derivatives thereof, a carotenoid, glycyrrhetinic, acid, glycyrrhizic acid or its salts, a centella extract or isolated ingredients thereof, a plectranthus extract, a boswellia extract, a ginger extract, an aloes extract, an angelica extract, an eleutherococcus extract, a rhodiola extract, an hippophae extract, a cyanotis extract, a vegetable oil, an oligopeptide, coenzyme Q10, ubiquinone, caffeine, theophylline, a tea extract, a cacao extract, a yeast extract, a soybean extract, a resveratrol and/or a procyanidin oligomer. Preferably that combination is contained in a topically dermatological or cosmetic composition.

Ginsenosides or the extract containing them according to the invention can also be combined with an agent for supporting the cosmetic qualities of skin and/or hair, supporting to stay slim, retaining muscular strength, improving memory, reducing cholesterol, reducing menopause side effects, preventing harmful effects of sunlight and/or preventing cardiovascular problems. Preferably that combination is contained in an orally applicable composition.

Ginsenosides or the extract containing them according to the invention can also be combined with polyunsaturated fatty acids or derivatives thereof, vitamins, oligoelements, a calcium salt, a carotenoid, a phytohormone, a polyphenol, a medicinal plant extract, a camosine and/or caffeine. Preferably that combination is contained in an orally applicable composition.

Ginsenosides or the extract containing them according to the invention can be used in the field of food supplement or in the dermatological field, which include cosmetically and pharmaceutically use, for the protection of the skin against deleterious effects of stress or irradiation e.g. sunlight or UV irradiation, preferably UV-A or UV-B irradiation.

Protection of the skin according to the invention include but is not limited to protection of the immune system of the skin, protection of the skin against oxidative or other stress, protection against inflammatory skin diseases, protection against apoptosis, protection against senescence, protection against hyperproliferation of skin cells and protection against imperfectly controlled respiration in mitochondriae.

Furthermore ginsenosides or the extract containing them according to the invention can be used for the protection of Langerhans cells, inducing an increase of HO-1 m-RNA expression in human skin fibroblasts, increasing the endogenous synthesis of heme oxygenase, increasing the endogenous production of carbon monoxide and bilirubin, preventing and/or limiting endogenous reactive oxygen species and/or eliminating reactive oxygen species from cells, preventing and/or limiting the photoimmunodepression in the skin e.g. caused by UV light exposure, preventing and/or limiting cellular apoptosis, senescence or loss of functionality of the cells, and/or for the treatment or prevention of diseases or conditions affected thereby.

Furthermore the ginsenosides or the extract containing them according to the invention can be used for wound healing, for skin regeneration and against skin aging.

### EXAMPLES

### Example 1

Roots or rhizomes of Panax notoginseng are extracted with ethanol. The fraction containing the ginsenosides is isolated by column-chromatography before spray-drying.

The corresponding product is in powder form and contains more than 90% of ginsenosides. The purity can be controlled by HPLC and shows extracts which can contain 10-60% of G-Rb1, 10-60% of G-Rg1, 0-15% of G-Rd, 0-15% of N-R1 and 0-10% G-Re by weight of the total extract.

### Example 2

The activity of the Panax notoginseng extract (according to Example 1) is evaluated in an ex vivo human skin model against UV-induced Langherans cell toxicity.

Skin punches are taken from abdominal skin biopsy and cultured in a medium composed by DMEM, Glutamine, penicillin-streptomycin and fetal calf serum. The Panax notoginseng extract diluted in DMSO is added in the culture medium twice, 24 h and one hour before UV-irradiation. Two other biopsies series without any treatment and with or without irradiation are prepared as controls with or without UV.

Langherans cells are then specifically labelled by AntiCD1a-FITC antibodies. Skin biopsies are frozen and sliced off. Then the sections are observed in epifluorescence in order to count the fluorescent Langherans cells.

The percentage of protection is calculated compared to control without UV according to the following formula:

$$P \% = \frac{( {Control}_{+ UV} - {Treated}_{- UV} ) \times 100}{{Control}_{- UV}}$$

In the control+UV the number of Langerhans cells located in the epidermis decreases by 95% and are labelled. A 24 h prior incubation with 0.04, 0.2 and 1 mg/ml of a Panax notoginseng root ginsenoside fraction prevents 32%, 55% and 63% of the decrease of Langerhans cells.

As the Panax notoginseng extract (according to Example 1) does not absorb UV A or UVB, the tested activity on Langherans cells is indeed linked to biological activity.

### Example 3

Gene expression is measured on cultivated human skin fibroblasts by cDNA array. Panax notoginseng extract (according to Example 1) is added at 0.2 mg/ml in DMSO to a monolayer culture. The test compound and the cells are incubated for 24 h in an assay medium. The analysis of genes expression is performed using standard minichips. The chips are spotted using a spotting device and cDNAs specific markers of interest.

It is found by a c-DNA array that the expression of the Heme Oxygenase-1 m-RNA is enhanced to 189% of the base level by a treatment with the ginsenoside fraction of Panax Notoginseng.

### Example 4

Activation of Heme-Oxygenase 1 by Panax notoginseng extract (according to Example 1) is determined by PCR (Polymerase Chains Reaction) amplification. Fibroblasts are cultured in an assay medium containing or not the test compound for 4 h, 8 h or 24 h. At the end of each incubation time, the cells are washed in PBS buffer, placed in Tri-reagent and frozen with G3PDH as reference.

The PCR is performed in triplicate using the LightCycler® system. The incorporation of fluorescence in amplified DNA was measured continuously during the PCR cycles. The ‘fluorescence intensity’ versus ‘PCR-cycle’ plot allows the evaluation of a relative expression value for each marker.

The expression of HO-1 m-RNA in the cells after 4 h of contact with the P.Notoginseng extract is 313% of the base level.

### Example 5

The evaluation of the protective effect of the Panax notoginseng extract (according to Example 1) against stress-induced premature senescence is tested in vitro on cultured human diploid fibroblasts. Stress-induced premature senescence can be defined as the long term effects of subtoxic stress on proliferative cell types and can be represented in vitro by H2O2-induction. The senescence-associated β-galactosidase is regarded as the biomarker of senescent, non-proliferative cells.

In the present study the fibroblasts treatment happens in 3 phases. In phase 1 fibroblasts are treated for 24 h with the culture medium containing the Panax notoginseng extract at three different concentrations of 50, 150 or 300 μg/ml (pre-treatment phase). Thereafter in phase 2 the medium is changed by a medium containing H2O2 (200 μM) for the stress induction. After 2 h the medium is changed again by the initial culture medium having again the three different concentrations of 50, 150 or 300 μg/ml (phase 3, recovery period). After 72 h recovery time the senescence-associated β-galactosidase is determined at pH 6 by colorimetric and fluorimetric tests.

According to the results exposure of fibroblasts to sub-toxic H₂O₂ dose leads to an increase of the amount of β-galactosidase positive cells from 17.6% to 43.5%. A concentration of 150 or 300 μg/ml in the culture medium 24 h before induction and after the period of recovery after the stress significantly decreases the percentage of stress-induced β-galactosidase positive cells, respectively to 37.5% and 33.2%.

### Example 6

### Tonifying Cream

| | Amount |
|---|---|
| INCI Name | [%] |
| Isononyl Isononanoate | 4.00 |
| Myristyl Alcohol (and) Myristyl Glucoside | 3.00 |
| Cyclomethicone | 3.00 |
| Glyceryl Stearate (and) PEG-100 Stearate | 2.00 |
| Dicaprylyl Ether | 2.00 |
| C12/15 Albyl Benzoate | 2.00 |
| Glycerin | 2.00 |
| Propylene Glycol | 1.00 |
| Ginsenosides (According to example 1) | 0.2 |
| Carbomer | 0.1 |
| Xanthan gum | 0.1 |
| Sodium Hydroxyde | Qs pH 5-6 |
| Water | Qs 100% |

1. A composition comprising of an active selected from the group consisting of ginsenosides and plant extracts containing ginsenosides, wherein said composition protects skin against deleterious effects of stress or irradiation.

2. The composition of claim 1, wherein said irradiation is selected from the group consisting of sunlight, UV-A radiation and UV-B radiation.

3. The composition of claim 1 wherein said composition protects skin against deleterious effects of stress or irradiation by at least one mechanism selected from the group consisting of: protecting Langerhans cells, inducing an increase of HO-1 m-RNA expression in human skin fibroblasts, increasing the endogenous synthesis of heme oxygenase, increasing the endogenous production of carbon monoxide and bilirubin, preventing endogenous reactive oxygen species, limiting endogenous reactive oxygen species, eliminating reactive oxygen species from cells, preventing the photoimmunodepression in the skin caused by UV light exposure, limiting the photoimmunodepression in the skin caused by UV light exposure, preventing cellular apoptosis, senescence or loss of functionality of the cells, limiting cellular apoptosis senescence or loss of functionality of the cells, and treating and preventing diseases or conditions affected thereby.

4. The composition of claim 1, wherein said composition protects skin against deleterious effects of stress or radiation by at least one mechanism selected from the group consisting of: protecting the immune system of the skin, protecting the skin against oxidative stress, protecting against inflammatory skin diseases, protecting against apoptosis, protecting against senescence, protecting against hyperproliferation of skin cells and protecting against imperfectly controlled respiration in mitochondriae.

5. The composition of claim 1 wherein said ginsenoside is selected from the group consisting of ginsenoside G-Ra1, G-Ra2, G-Ra3, G-Rb1, G-Rb2, G-Rb3, G-Rc, G-Rd, G-Re, G-Rf, G-Rg1, G-Rg2, G-Rg3, G-Rh1, G-Rh2, G-Rs1, G-Rs2, G-Ro, MG-Rb1, MG-Rb2, MG-Rc, MG-Rd, Q-R1, N-R1, N-R4, 20 Glc-Rf or mixtures thereof.

6. The composition of claim 1, wherein said plant extract comprises at least one extract selected from the group consisting of: an extract of Panax notoginseng (San Qi), Panax ginseng (C. A. Meyer) and Panax quinquefolium.

7. The composition of claim 6, wherein said at least one extract is comprises an extract of the roots or the rhizomes of Panax notoginseng (San Qi), Panax ginseng (C. A. Meyer) or Panax quinquefolium.

8. The composition of claim 1, wherein said extract comprises from about 0.1% to about 100% ginsenosides by weight of the total extract.

9. The composition of claim 8, wherein said extract comprises not less than about 80% ginsenosides by weight of the total extract.

10. The composition of claim 1, wherein extract comprises from about 10% to about 60% G-Rb1, G-Rg1 in an amount of from about 10% to about 60% G-Rg1, from about 0% to about 15% G-Rd, from about 0% to about 15% N-R1, and from about 0% to about 10% G-Rd, all by weight of the total extract.

11. The composition of claim 1, wherein said composition comprises from about 0.01% to about 10% ginsenosides by weight of the total composition.

12. The composition of claim 1, wherein said composition comprises from about 0.001% to about 6% G-Rb1, from about 0.001% to about 6% G-Rg1, about 0% to about 1.5% G-Rd, from about 0% to about 1.5% N-R1 and from about 0% to about 1% G-Re, all by weight of the total composition.

13. The composition of claim 1, further comprising at least one additional active substance or plant extract.

14. The composition of claim 13, wherein said at least one additional active substance or plant extract comprises an active substance or plant extract having at least one dermatological use.

15. The composition of claim 1, wherein said composition is a dermatological or cosmetic composition for topical administration.

16. The composition of claim 1, wherein said composition comprises a liquid solution or a cream.

17. The composition of claim 1, wherein said composition comprises a food supplement.

18. The composition of claim 17, wherein said food supplement is selected from the group consisting of: a normal and enteric coated tablets, capsules, pills, granules, elixirs, solutions or suspensions.
